(19) United States
(12) Patent Application Publication
(10) Pub. No.: US 2026/0314603 A1
(43) Pub. Date: Oct. 15, 2026

(54) METHOD FOR REDUCING PLANT STRESS, FUNGI, AND MYCOTOXINS

(71) Applicants: UNIVERSIDAD POLITÉCNICA DE MADRID, Madrid (ES); PLANT RESPONSE, INC., Durham, NC (US)

(72) Inventors: Maria Soledad SACRISTÁN BENAYAS, Madrid (ES); Sandra DÍAZ GONZÁLEZ, Madrid (ES); Carlos GONZÁLEZ SANZ, Madrid (ES); Palmira DEL PRADO POLONIO, Madrid (ES); Frederic BRUNNER, Madrid (ES); Patricia MARÍN GARCÍA, Madrid (ES); Anne RUNKEL, Durham, NC (US); Rosa Maria PERÉZ JIMENEZ, Madrid (ES)

(21) Appl. No.: 19/122,600

(22) PCT Filed: Oct. 18, 2023
(86) PCT No.: PCT/US2023/077169

§ 371 Date: Apr. 18, 2025

(30) Foreign Application Priority Data
Oct. 18, 2022 (ES) P202230895

(51) Int. Cl. A01N 63/30; A01P 21/00

(52) U.S. Cl. CPC A01N 63/30; A01P 21/00

(57) ABSTRACT

Methods of treating a plant or part thereof, or a growth medium for growing a plant, with a composition comprising an effective amount or rate of Colletotrichum tofieldiae to increase abiotic and/or biotic stress tolerance/resistance of the plant or part thereof. For example, a method for reducing fungal genera related to mycotoxin production, can include contacting said plant with a composition comprising the microorganism Colletotrichum tofieldiae and/or extracts from said microorganism and/or filtrates from said microorganism. A plant is contacted with a composition comprising the microorganism C. tofieldiae, the culture medium or the filtrate. The composition can be applied to the entire plant or to any of the parts thereof, such as to the leaves, sprouts, flowers, fruits, cobs, seeds, bulbs, tubers, roots and to the seedlings. Application of the composition to the plant can be carried out at any stage in the planting, growing, and/or harvesting season.

### RELATED APPLICATIONS

This application claims priority to Spanish Application No. 202230895, filed Oct. 18, 2022, which is incorporated herein by reference in its entirety.

### TECHNICAL FIELD

The present disclosure relates to the agronomic field, particularly methods for reducing plant stress, including abiotic and biotic, plant and plant-product colonization by fungi, and mycotoxin producing fungi using the microorganism Colletotrichum tofieldiae.

### BACKGROUND

Plants in nature establish symbiotic associations with microorganisms referred to as mutualists conferring benefits on the growth, survival, and multiplication thereof. These microorganisms can be isolated, and their beneficial properties can be used in crops so as to improve yield thereof.

The genus Colletotrichum (Ascomycete, teleomorph Glomerella) comprises more than 60 species and species complexes. It is morphologically characterized by typically acervular conidiomata, with or without setae, having unicellular hyaline conidia which can be straight or curved, with a size preferably bigger than 12μ, usually granular. Conidia can also be formed from mycelium or other conidia (microcyclic conidia). Conidia form appressoria when they germinate. Some species form stromas or sclerotia.

The genus Colletotrichum comprises species which are important crop pathogens, and which are, because of that, the most common and best studied species. However, within the genus there are many species which have been reported as being endophytes or epiphytes which cause no damage to the host plant (referred to as commensal), or which can even be beneficial for the plant (mutualists). Hyde et al. (Fungal Diversity 39 (2009) 147-182) provides a thorough description of all the species from the genus Colletotrichum which are currently known, including a list of hosts cited in the literature and specifying the type of interactions established with each host (pathogenic, commensal or mutualist). The evidence cited in this publication indicates that, among the species or species complexes considered as pathogenic, there may be asymptomatic strains. There are even cases of strains which behave as pathogens, commensals, or mutualists depending on the host in which they are inoculated. An example of this case is C. orbiculare, which behaves as a pathogen in curcubitaceae but may behave as a mutualist in tomatoes, conferring resistance to pathogens and to drought and promoting the vegetative growth of the plant when it is root inoculated.

### SUMMARY OF THE DISCLOSURE

The inventors of the present disclosure have discovered that treating a plant and/or part thereof, and/or a growth medium for growing a plant, with a composition comprising an effective amount/rate of Colletotrichum tofieldiae can increase abiotic and/or biotic stress tolerance/resistance of the plant and/or part thereof.

An embodiment of the disclosure is a method for reducing fungal genera related to mycotoxin production, which can include contacting said plant with a composition comprising the microorganism Colletotrichum tofieldiae and/or extracts from said microorganism and/or filtrates from said microorganism, hereinafter referred to as “method of the disclosure”.

Hereinafter, the term “microorganism of the disclosure” refers to the microorganism from the species Colletotrichum tofieldiae.

Mycotoxins are metabolites produced by fungi. Mycotoxin producing fungi may grow on plants and plant-derived products. Mycotoxin contamination of plants and plant-derived products can have negative impacts on agronomic production, crop quality, and pose risks to those who consume the plants or and plant-derived products. Reducing the fungal genera related to mycotoxin production with the method of the disclosure should be understood as a decrease in the various genera that produce mycotoxins and cause post-harvest diseases in maize grains compared to a plant that has not been treated according to the method of the disclosure.

The species C. tofieldiae is described in Damm et al. (Fungal Diversity 39 (2009) 45-87). This species is characterized by curved conidia and acervuli with setae which can be brown or black coloured. Conidia and setae can be formed directly over hyphae. Spores germinate forming appressoria featuring varied morphology and colouring. The definition of the species is also due to molecular characteristics with respect to sequences from the ribosome 5.8S subunit having the two flanking spacer regions (ITS), a 200-bp intron from the Glyceraldehyde 3-phosphate dehydrogenase (GAPDH) gene, a partial sequence from the actin (ACT) gene, chitin synthase 1 (CHS-1) gene, beta-tubulin (Tub2) gene and histone 3 (HIS3) gene. The typical isolates used to describe the species C. tofieldiae come from the tofieldia spp. (monocotyledons), Lupinus polyphyllus and Dianthus sp. (both dicotyledoneae), therefore this species can colonize different species of hosts both from mono and dicotyledoneae. C. tofieldiae has not been reported as pathogenic for any host.

In the present disclosure, isolates of C. tofieldiae have been isolated, identified and characterized, which have the ability to significantly decrease fungal colonization, including those related to mycotoxin production and post-harvest diseases in maize grains. Citing Díaz-González et al. 2020 Agronomy 2020, 10, 1493; doi: 10.3390/agronomy 10101493, incorporated herein by reference in its entirety, it is shown that the fungus can be reisolated from different parts or tissues of the of plants (see page 7). For example, it can be detected in maize roots at harvest (Id. at page 9), and can be detected in tomato roots and or stems as soon as one month after inoculation of a tomato plant.

The method of disclosure decreases fungal colonization, particularly of the mycotoxin-producing genera, especially within Aspergillus spp. It is the first time this feature is described in the C. tofieldiae strains of the disclosure, and it is a phenomenon being general to the species C. tofieldiae. In the method of the disclosure, a plant is contacted with a composition comprising the microorganism C. tofieldiae, the culture medium or the filtrate according to the disclosure. The composition can be applied to the entire plant or to any of the parts thereof, such as to the leaves, sprouts, flowers, fruits, cobs, seeds, bulbs, tubers, roots and to the seedlings. Application of the composition to the plant can be carried out at any stage, for example, it can be applied to the seed before the sowing, during the sowing, after the sowing, and before or after the shoot emergence, during the growing season, such as during seedbed cultivation, or at the time the seedlings are transplanted, or when plant cuttings or rooting of the cuttings take place, or at the growing period in a plantation, at the reproductive stage before flowering or during flowering or during the ripening process of the fruit, or after the fruit or grain has been harvested.

The present disclosure has applications in the agronomic sector. One embodiment provides methods and compositions for increasing biotic and/or abiotic stress tolerance or resistance in a plant and/or part thereof comprising applying a composition comprising C. tofieldiae to a plant and/or part thereof and/or to the media in which a plant and/or part thereof is growing.

Therefore, another embodiment refers to the method of the disclosure wherein said composition is applied to the seeds of said plant.

Another embodiment refers to the method of the disclosure wherein said composition is applied to aerial parts of said plant.

Another embodiment refers to the method of the disclosure wherein said composition is applied to the roots of or the soil surrounding said plant or to other underground parts of said plant.

The method of the disclosure includes spray or atomization treatment over the entire plant or any part or plant product thereof, with an appropriate dilution of the composition according to the disclosure, or immersion of the entire plant or of any part thereof in said dilution. The method of the disclosure also includes dry dusting treatment of the entire plant or of any part thereof with the composition according to the disclosure. The method of the disclosure includes seed application, pellet applications, or coating the plant or soil surface using a thin layer of a composition according to the disclosure. The composition according to the disclosure can also be mixed with the irrigation liquid. The method of the disclosure also includes treatment by means of mycelium hyphae, spores, or sclerotia, which are contacted with one part of the plant, such as the root, stem or leaves, or the soil surface near the roots of the crop.

In the present description, the term “filtrate” means a liquid culture medium obtained from growing the microorganism of the disclosure. It is possible to obtain a liquid culture medium being free or essentially free from the microorganism of the disclosure. The culture medium may be firstly prepared by growing the microorganism of the disclosure in a liquid culture medium, and then separating the liquid culture medium from the microorganism of the disclosure. Separation may be carried out by means of different methods well known to those skilled in the art, for example, by centrifugation or filtration. It is possible, for example, to heat the medium with the microorganism of the disclosure twice, up to about 80 °C for 30 min and then remove the fungal material by centrifugation.

The filtrate can be obtained by filtration of the culture medium through a filter having a pore size of no more than 2 μm, preferably through a filter having a pore size of no more than 0.2 μm. The filtration step allows removal of essentially all hyphae from the microorganism of the disclosure, more preferably the filtration should also remove spores, and even more preferably it should remove any type of fungal material.

In the present description, the term “extract” means a purification, dehydration, or isolation of components from a culture obtained from growing the microorganism of the disclosure. It is possible to obtain an extraction medium being free or essentially free from the microorganism of the disclosure. An extraction can also include components of the microorganism of the disclosure. Methods of extraction are well known to those skilled in the art, for example, using grinding, lyophilization, solvents, distillation, pressure, heat, liquid chromatography, liquid chromatography-tandem mass spectrometry (LC-MS-MS), and/or gas chromatography-mass spectrometry (GC-MS). The present disclosure also refers to plants that, according to the method of the disclosure, have been contacted with a composition comprising the microorganism C. tofieldiae and/or extracts from said microorganism and/or filtrates from said microorganism, and/or to products produced from the harvested parts of said plants.

In some embodiments, a composition may comprise an amount of the microorganism C. tofieldiae and/or extracts from said microorganism and/or filtrates from said microorganism in a range from about 0.01 mg per liter (L) to about 100 g per liter of the composition. Thus, in some embodiments, a composition of the disclosure may comprise the microorganism C. tofieldiae and/or extracts from said microorganism and/or filtrates from said microorganism in a range from about 0.01 mg/L to about 1 mg/L, about 0.01 mg/L to about 10 mg/L, about 0.01 mg/L to about 100 mg/L, about 0.01 mg/L to about 1 g/L, about 0.01 mg/L to about 10g/L, about 0.01 mg/L to about 100 g/L, about 0.01 g/L to about 1 g/L, about 0.01 g/L to about 5 g/L, about 0.01 g/L to about 10 g/L, about 0.1 g/L to about 15 g/L, about 0.01 g/L to about 20 g/L, about 0.01 g/L to about 30 g/L, about 0.01 g/L to about 40 g/L, about 0.01 g/L to about 100 g/L, about 0.5 g/L to about 1 g/L, about 0.5 g/L to about 5 g/L, about 0.5 g/L to about 10 g/L, about 0.5 g/L to about 20 g/L, about 0.5 g/L to about 30 g/L, about 0.5 g/L to about 40 g/L, about 0.5 g/L to about 50 g/L, about 0.5 g/L to about 100 g/, about 1 g/L to about 5 g/L, about 1 g/L to about 10 g/L, about 1 g/L to about 15 g/L, about 1 g/L to about 20 g/L, about 1 g/L to about 30 g/L, about 1 g/L to about 40 g/L, about 1 g/L to about 50 g/L, about 1 g/L to about 100 g/L, about 5 g/L to about 10 g/L, about 5 g/L to about 15 g/L, about 5 g/L to about 20 g/L, about 5 g/L to about 30 g/L, about 5 g/L to about 40 g/L, 1, about 5 g/L to about 50 g/L, about 5 g/L to about 100 g/L, about 10 g/L to about 15 g/L, about 10 g/L to about 20 g/L, about 10 g/L to about 30 g/L, about 10 g/L to about 40 g/L, about 10 g/L to about 50 g/L, about 10 g/L to about 100 g/L, about 15 g/L to about 20 g/L, about 15 g/L to about 30 g/L, about 15 g/L to about 40 g/L, about 15 g/L to about 50 g/L, about 15 g/L to about 100 g/L, about 20 g/L to about 30 g/L, or about 20 g/L to about 40 g/L, about 20 g/L to about 50 g/L, about 20 g/L to about 100 g/L, about 30 g/L to about 40 g/L, about 30 g/L to about 50 g/L,, about 30 g/L to about 100 g/L, about 40 g/L to about 50 g/L or about 40 g/L to about 100 g/L of the composition, or any value or range therein.

In some embodiments, an effective amount of the microorganism C. tofieldiae and/or extracts from said microorganism and/or filtrates from said microorganism is an amount sufficient to increase abiotic stress and/or biotic stress tolerance/resistance of a plant and/or part thereof. In some embodiments, an effective amount of the microorganism C. tofieldiae and/or extracts from said microorganism and/or filtrates from said microorganism in a composition may be from about 0.01 mg per liter to about 100 g per liter of the composition. In some embodiments, an effective amount of the microorganism C. tofieldiae and/or extracts from said microorganism and/or filtrates from said microorganism in a composition may be from about 0.01 mg/L to about 1 mg/L, about 0.01 mg/L to about 10 mg/L, about 0.01 mg/L to about 100 mg/L, about 0.01 mg/L to about 1 g/L, about 0.01 mg/L to about 10g/L, about 0.01 mg/L to about 100 g/L, about 0.01 g/L to about 1 g/L, about 0.01 g/L to about 5 g/L, about 0.01 g/L to about 10 g/L, about 0.01 g/L to about 15 g/L, about 0.01 g/L to about 20 g/L, about 0.01 g/L to about 30 g/L, about 0.01 g/L to about 40 g/L, about 0.01 g/L to about 100 g/L, about 0.5 g/L to about 1 g/L, about 0.5 g/L to about 5 g/L, about 0.5 g/L to about 10 g/L, about 0.5 g/L to about 20 g/L, about 0.5 g/L to about 30 g/L, about 0.5 g/L to about 40 g/L, about 0.5 g/L to about 50 g/L, about 0.5 g/L to about 100 g/, about 1 g/L to about 5 g/L, about 1 g/L to about 10 g/L, about 1 g/L to about 15 g/L, about 1 g/L to about 20 g/L, about 1 g/L to about 30 g/L, about 1 g/L to about 40 g/L, about 1 g/L to about 50 g/L, about 1 g/L to about 100 g/L, about 5 g/L to about 10 g/L, about 5 g/L to about 15 g/L, about 5 g/L to about 20 g/L, about 5 g/L to about 30 g/L, about 5 g/L to about 40 g/L, 1, about 5 g/L to about 50 g/L, about 5 g/L to about 100 g/L, about 10 g/L to about 15 g/L, about 10 g/L to about 20 g/L, about 10 g/L to about 30 g/L, about 10 g/L to about 40 g/L, about 10 g/L to about 50 g/L, about 10 g/L to about 100 g/L, about 15 g/L to about 20 g/L, about 15 g/L to about 30 g/L, about 15 g/L to about 40 g/L, about 15 g/L to about 50 g/L, about 15 g/L to about 100 g/L, about 20 g/L to about 30 g/L, or about 20 g/L to about 40 g/L, about 20 g/L to about 50 g/L, about 20 g/L to about 100 g/L, about 30 g/L to about 40 g/L, about 30 g/L to about 50 g/L, about 30 g/L to about 100 g/L, about 40 g/L to about 50 g/L or about 40 g/L to about 100 g/L of the composition, or any value or range therein.

In some embodiments, a composition comprising the microorganism C. tofieldiae and/or extracts from said microorganism and/or filtrates from said microorganism may further comprise a peptide, a protein, a sugar and/or a carbohydrate. In some embodiments, the microorganism C. tofieldiae and/or extracts from said microorganism and/or filtrates from said microorganism composition may comprise peptides and/or proteins in an amount from about 0.1% to about 10% w/w of the extract. In some embodiments, a composition comprising the microorganism C. tofieldiae and/or extracts from said microorganism and/or filtrates from said microorganism comprise peptides and/or proteins in an amount from about 0.1% to about 1%, about 0.1% to about 3%, about 0.1% to about 5%, about 0.1% to about 7%, about 0.5% to about 1%, about 0.5% to about 3%, about 0.5% to about 5%, about 0.5% to about 7%, about 0.5 to about 10%, about 1% to about 3%, about 1% to about 5%, about 1% to about 7%, about 1% to about 10%, about 3% to about 5%, about 3% to about 7%, about 3% to about 10%, about 5% to about 7%, about 5% to about 10%, or about 7% to about 10%, or any range or value therein of the composition. Thus, in some embodiments, a composition comprising the microorganism C. tofieldiae and/or extracts from said microorganism and/or filtrates from said microorganism may comprise peptides and/or proteins in an amount of about 0.01, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, or 10% w/w or any range or value therein of the composition.

In some embodiments, a composition comprising the microorganism C. tofieldiae and/or extracts from said microorganism and/or filtrates from said microorganism may comprise other sugars and/or carbohydrates in an amount from about 1% to about 35% w/w of the extract. In some embodiments, a composition comprising the microorganism C. tofieldiae and/or extracts from said microorganism and/or filtrates from said microorganism may comprise other sugars and/or carbohydrates in an amount from about 1% to about 5%, about 1% to about 10%, about 1% to about 15%, about 1% to about 20%, about 1% to about 25%, about 1% to about 30%, about 5% to about 10%, about 5% to about 15%, about 5% to about 20%, about 5% to about 25%, about 5% to about 30%, about 5% to about 35%, about 10% to about 15%, about 10% to about 20%, about 10% to about 25%, about 10% to about 30%, about 10% to about 35%, about 15% to about 20%, about 15% to about 25%, about 15% to about 30%, about 15% to about 35%, about 20% to about 25%, about 20% to about 30%, about 20% to about 35%, about 25% to about 30%, about 25% to about 35%, or about 30% to about 35% w/w of the extract, or any value or range therein. Thus, in some embodiments, a composition comprising the microorganism C. tofieldiae and/or extracts from said microorganism and/or filtrates from said microorganism may comprise additional sugars and/or carbohydrates in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35% w/w of the extract or any range or value therein.

In some embodiments, sugars and/or carbohydrates that may be comprised in a composition comprising the microorganism C. tofieldiae and/or extracts from said microorganism and/or filtrates from said microorganism can include, but is not limited to glucose, mannose, galactose, arabinose, xylose other glucan oligosaccharides, glucose-derived low branched polysaccharides, glycogen, mannan oligosaccharides, mannose-derived low branched polysaccharides, galactans, galactomannans, arabinans and/or xylans.

In some embodiments, a composition comprising the microorganism C. tofieldiae and/or extracts from said microorganism and/or filtrates from said microorganism may further comprise additional components including, but not limited to, a surfactant, a humectant, an adjuvant, an antioxidant, a preservative, a plant macronutrient, a plant micronutrient, a plant growth regulator, a pesticide, a fungicide, an antiviral, an anti-bacterial, an herbicide, or any combination thereof.

Example surfactants can include, but are not limited to, alkali metal, alkaline earth metal and ammonium salts of ligno-sulfonic acid, naphthalenesulfonic acid, phenolsulfonic acid, dibutylnaphthalenesulfonic acid, alkylarylsulfonates, sodium dodecylsulfate, alkyl sulfates, alkylsulfonates, fatty alcohol sulfates, fatty acids and sulfated fatty alcohol glycol ethers, of sulfonated condensates naphthalene and naphthalene derivatives with formaldehyde, condensates of naphthalene or of naphthalenesulfonic acid with phenol and formaldehyde, polyoxyethylene octyl-phenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol, polyglycol ether acetal, sorbitol esters, lignin-sulfite waste liquors and/or methylcellulose.

In some embodiments, a surfactant may be present in a composition comprising the microorganism C. tofieldiae and/or extracts from said microorganism and/or filtrates from said microorganism in an amount from about 0.1% to about 40% w/w of the composition. In some embodiments, a surfactant may be present in a composition comprising the microorganism C. tofieldiae and/or extracts from said microorganism and/or filtrates from said microorganism in an amount from about 0.1% to about 1%, about 1% to about 10%, about 10% to about 20%, about 10% to about 25%, about 10% to about 30%, about 10% to about 35%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, about 35% to about 40% w/w of the composition or any range or value therein). Thus, in some embodiments, the surfactant may be present in a composition comprising the microorganism C. tofieldiae and/or extracts from said microorganism and/or filtrates from said microorganism in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.5%, 3%, 3.5%, 4%, 4.5%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40% w/w of the composition or any range or value therein.

Example humectants can include, but are not limited to, glycerol, sorbitol, xylitol, maltitol, glyceryl triacetate, sodium lactate, urea formaldehyde, propylene glycol, ethylene glycol and/or fatty acids.

An example antioxidant can include, but is not limited to, ascorbic acid, tocopherols, propyl gallate, tertiary butylhydroquinone, butylated hydroxyanisole, and/or butylated hydroxytoluene.

An example preservative can include, but is not limited to, sorbic acid, sodium sorbate, sorbates, benzoic acid, sodium benzoate, benzoates, hydroxybenzoate and derivatives, sulfur dioxide and sulphites, nitrite, nitrate, lactic acid, propionic acid and sodium propionate, tocopherol, plant extract, hops, salt, sugar, vinegar, alcohol (e.g. methanol and ethanol), diatomaceous earth and castor oil, citric acid, ascorbic acid, sodium ascorbate, phenol derivatives (butylated hydroxytoluene, butylated hydroxyanisole, BHA, BHT, TBHQ and propyl gallate), gallic acid, sodium gallate, sulfur dioxide, sulphites, tocopherols, and/or methylchloroisothiazolinone, 1,2-Benzisothiazolin-3-one (BIT), Hexahydro-1,3,5-tris-hydroxyethyl-s-triazine (HTHT), 5-chloro-2-methyl-2H-isothiazol-3-one (CMIT), 2-methyl-2H-isothiazol-3-one (MIT), Zinc pyrithione (ZPT), 2-Bromo-2-nitropropane-1,3-diol (Bronopol), Formaldehyde, 1,3-Dimethylol-5,5-dimethylhydantoin (DMDMH), 2,2-Dibromo-3-nitrilopropionamide (DBNPA), and/or Poly (hexamethylene biguanide) hydrochloride (PHMB).

In some embodiments, a preservative may be present in a composition comprising the microorganism C. tofieldiae and/or extracts from said microorganism and/or filtrates from said microorganism in an amount ranging from about 0.001% to about 5% w/w or any range or value therein. In some embodiments, a composition may comprise a preservative in an amount ranging from about 0.001% to about 0.1%, about 0.001% to about 0.5%, about 0.001% to about 1%, about 0.001% to about 2%, about 0.001% to about 3%, about 0.001% to about 4%, about 0.01% to about 0.1%, about 0.01% to about 0.5%, about 0.01% to about 1%, about 0.01% to about 2%, about 0.01% to about 3%, about 0.01% to about 4%, about 0.01% to about 5%, about 0.05% to about 0.1%, about 0.05% to about 0.5%, about 0.05% to about 1%, about 0.05% to about 2%, about 0.05% to about 3%, about 0.05% to about 4%, about 0.05% to about 5%, about 0.1% to about 0.5%, about 0.1% to about 1%, about 0.1% to about 2%, about 0.1% to about 3%, about 0.1% to about 4%, about 0.1% to about 5%, about 0.5% to about 1%, about 0.5% to about 2%, about 0.5% to about 3%, about 0.5% to about 4%, about 0.5% to about 5%, about 1% to about 2%, about 1% to about 3%, about 1% to about 4%, about 1% to about 5%, about 2% to about 3%, about 2% to about 4%, about 4% to about 5%, about 3% to about 4%, about 3% to about 5%, about 4% to about 5% w/w of the composition, or any range or value therein. Thus, in some embodiments, the preservative may be present in a composition comprising the microorganism C. tofieldiae and/or extracts from said microorganism and/or filtrates from said microorganism in an amount of about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.5%, 3%, 3.5%, 4%, 4.5%, 5% w/w of the composition or any range or value therein.

Example plant macronutrients include, but are not limited to, nitrogen, potassium, calcium, magnesium, phosphorus, and/or sulfur.

Example plant micronutrients can include, but are not limited to, iron, manganese, boron, molybdenum, copper, zinc, chlorine, and/or cobalt.

Example plant growth regulators include, but are not limited to, auxin (including but not limited to naphthalene acetic acid (NAA) and/or indole-3-butyric acid (IBA) and/or indole-3-acetic acid (IAA, 3-IAA)), cytokinin, abscisic acid, gibberellin, ethylene, salicylic acid, jasmonic acid, brassinosteriod (e.g., brassinolide), or any combination thereof.

Example pesticides include, but are not limited to, malathion, parathion, methyl parathion, chlorpyrifos, diazinon, dichlorvos, phosmet, fenitrothion, tetrachlorvinphos, azamethiphos, fenvalerate, cyfluthrin, lambda-cyhalothrin, zeta-cypermethrin, permethrin, piperonyl butoxide, imidacloprid, acetamiprid, clothianidin, nitenpyram, nithiazine, thiacloprid, thiamethoxam, ryanodol, 9,21-didehydroryanodol, chlorantraniliprole, flubendiamide, and/or cyantraniliprole.

Example fungicides include, but are not limited to, prothioconazole, trifloxystrobin, azoxystrobin, propiconazole, and/or pyraclostrobin.

Example anti-bacterials (bactericides) include, but are not limited to, methylisothiazolinone, chloromethylisothiazolinone, benzisothiazolinone, octylisothiazolinone, dichlorooctylisothiazolinone, and/or butylbenzisothiazolinone

Example herbicides can include, but are not limited to, glyphosate, 2,4-dichlorophenoxyacetic acid, atrazine, S-metolachlor, and/or 3,6-dichloro-2-methoxybenzoic acid.

In some embodiments, a composition comprising the microorganism C. tofieldiae and/or extracts from said microorganism and/or filtrates from said microorganism may further comprise an antifoaming agent. Any antifoaming agent for use with agricultural and/or food products may be used. Example antifoaming agents include, but are not limited to, long chain unsaturated fatty acids including, but not limited to C12 to C14, C18:1 and C18:2 unsaturated fatty acids, and/or synthetic polysiloxanes (silicones) including, but not limited to, polydimethylsiloxane, and/or hydrophobic silica. In some embodiments, a composition comprising the microorganism C. tofieldiae and/or extracts from said microorganism and/or filtrates from said microorganism may comprise an amount of antifoaming agent in a range from about 0.0001% to about 0.05% w/w of the composition or any range or value therein. Thus, in some embodiments, the antifoaming agent may be present in the composition in an amount of about 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, or 0.05% w/w of the composition or any range or value therein.

In some embodiments, a composition comprising the microorganism C. tofieldiae and/or extracts from said microorganism and/or filtrates from said microorganism may further comprise a biocide. Any biocide for use with agricultural and/or food products may be used. When included in a composition comprising the microorganism C. tofieldiae and/or extracts from said microorganism and/or filtrates from said microorganism, a biocide may be present at about 0.1 g L⁻¹ to about 20 g L⁻¹ of the composition, or any range or value therein. Thus, in some embodiments, the biocide may be present in the composition in an amount of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2, 3, 4, 5, 6, 7, 8, 9, 10, 11, 12, 13, 14, 15, 16, 17, 18, 19, or 20 g per liter of the composition, or any range or value therein.

In some embodiments, the present disclosure provides a method for increasing disease tolerance of a plant and/or part thereof, the method comprising applying a composition comprising an effective amount of the microorganism C. tofieldiae and/or extracts from said microorganism and/or filtrates from said microorganism to a plant and/or a plant part thereof, and/or to a growth media, thereby increasing the disease tolerance of the plant and/or part thereof as compared to a control plant and/or part thereof (e.g., a plant and/or part thereof to which a composition comprising the microorganism C. tofieldiae and/or extracts from said microorganism and/or filtrates from said microorganism has not been applied). In some embodiments, the method comprises applying the composition at least one time (e.g., about 1, 2, 3, 4, 5, 6, 7, 8, 9, 10, 11, 12 or more times). In some embodiments, the method comprises applying the composition at least two times (e.g., about 2, 3, 4, 5, 6, 7, 8, 9, 10, 11, 12 or more times).

In some embodiments, when a composition comprising the microorganism C. tofieldiae and/or extracts from said microorganism and/or filtrates from said microorganism is applied at least two times to a plant and/or part thereof, and/or growth media, the range of time between application may vary. Thus, for example, a subsequent application of a composition comprising the microorganism C. tofieldiae and/or extracts from said microorganism and/or filtrates from said microorganism may be any time from about 1 day to about six months after the prior application. Thus, for example, a subsequent application may be applied about 1, 2, 3, 4, 5, 6 days or about 1, 2, 3, 4, 5, 6, 7, 8, 9, 10, 11, 12, 13, 14, 15, 16, 17, 18, 19, 20, 21, 22, 23, 24, 25, or 26 weeks after previous application, or any range or value therein.

In some embodiments, a composition comprising the microorganism C. tofieldiae and/or extracts from said microorganism and/or filtrates from said microorganism applied to a plant and/or part thereof, and/or to a growth media, may increase disease tolerance or resistance to a viral pathogen including, but not limited to, a virus from the virus family of Caulimoviridae, Potyviridae, Sequiviridae, Rheoviridae, Capillovirus, Geminiviridae, Bromoviridae, Closteroviridae, Comoviridae Tombusviridae, Rhabdoviridae, Bunyaviridae, Partitiviridae, Carlavirus, Enamovirus, Furovirus, Hordeivirus, Idaeovirus, Luteovirus, Marafivirus, Potexvirus, Sobemovirus, Tenuivirus, Tobamovirus, Tobravirus, Trichovirus, Tymovirus and/or Umbravirus.

In some embodiments, a composition comprising the microorganism C. tofieldiae and/or extracts from said microorganism and/or filtrates from said microorganism applied to a plant and/or part thereof, and/or to a growth media, may increase resistance to a virus, including but not limited to, turnip mosaic virus, papaya ring spot virus, bud blight virus, bean pod mottle virus, lettuce mosaic virus, maize mosaic virus, cauliflower mosaic virus, tobacco mosaic virus, soybean mosaic virus, African cassava mosaic virus, tomato mosaic virus, pepino mosaic virus, zucchini yellow mosaic virus, plum pox virus, tomato bushy stunt virus, tomato spot wilt virus, tomato yellow leaf curl virus, rice ragged stunt virus, rice tungro bacilliform, virus, rice tungro spherical virus, rice yellow mottle virus, cucumber mosaic virus, brome mosaic virus, wheat yellow mosaic virus, barley yellow dwarf virus, sugarcane mosaic virus, beet yellows virus, lettuce yellows virus, maize dwarf mosaic virus, maize streak virus, peanut stunt virus, Citrus tristeza virus, potato leafroll virus, potato virus X, potato virus Y, sweet potato feathery mottle potyvirus, Melon necrotic spot virus, maize white line mosaic virus, maize chlorotic mottle virus, banana bunchy top virus, cacao swollen shoot virus, tomato leaf curl New Dehli virus, banana streak virus, and/or sweet potato sunken vein closterovirus.

In some embodiments, a composition comprising the microorganism C. tofieldiae and/or extracts from said microorganism and/or filtrates from said microorganism applied to a plant and/or part thereof, and/or to a growth media, may increase resistance to a fungal pathogen including, but not limited to, a fungal/oomycete pathogen from the family of Physodermataceae, Synchytriaceae, Olpidiaceae, Choanephoraceae, Gilbertellaceae, Mucoraceae, Dipodascaceae, Eremotheciaceae, Taphrinaceae, Botryosphaeriaceae, Capnodiaceae, Phaeosphaeriaceae, Leptosphaeriaceae, Cucurbitariaceae, Didymellaceae Davidiellaceae, Mycosphaerellaceae, Schizothyriaceae, Dothideaceae, Dothioraceae, Lahmiaceae, Elsinoaceae, Lophiostomataceae, Pleosporaceae, Venturiaceae, Trichochomaceae, Erysiphaceae, Cyttariaceae, Hemiphacidiaceae, Hyaloscyphaceae, Phacidiaceae, Sclerotiniaceae, Ascodichaenaceae, Mediolariaceae, Rhytismataceae, Meliolaceae, Caloscyphaceae, Sarcosomataceae, Cryphonectriaceae, Diaporthaceae, Gnomoniaceae, Valsaceae, Glomerellaceae, Plectosphaerellaceae, Bionectriaceae, Clavicipitaceae, Hypocreaceae, Nectriaceae, Magnaporthaceae, Pyriculariaceae, Ceratocystideae, Ophiostomataceae, Phyllachoraceae, Chaetomiaceae, Amphisphaeriaceae, Diatrypaceae, Xylariaceae, Psathyrellaceae, Marasmiaceae, Mycenaceae, Schizophyllaceae, Typhulaceae, Thelephoraceae, Atheliaceae, Atheliaceae, Stereaceae, Echinodontiaceae, Corticiaceae, Ganodermataceae, Hymenochaetaceae, Cystofilobasidiaceae, Helicobasidiaceae, Helicobasidiaceae, Melampsoraceae, Phakopsoraceae, Pucciniaceae, Tilletiaceae, Entylomataceae, Ustilaginaceae, Leptolegniaceae and/or Peronosporaceae.

In some embodiments, a composition comprising the microorganism C. tofieldiae and/or extracts from said microorganism and/or filtrates from said microorganism applied to a plant and/or part thereof, and/or to a growth media, may increase resistance to a fungal pathogen including, but not limited to, Physoderma alfalfa, Physoderma maydis, Synchytrium endobioticum, Olpidium brassicae, Choanephora cucurbitarum, Mucor circinelloides, Rhizopus stolonifera, Geotrichum candidum, Taphrina caerulescens, Taphrina deformans, Taphrina populina, Botryosphaeria dothidea, Diplodia mutila, Dothiorella sarmentorum, Macrophomina phaseolina, Phyllosticta ampelicida, Phyllosticta citricarpa, Stenocarpella maydis, Cladosporium allii-cepae, Cladosporium cladosporioides, Acrodontium simplex, Cercospora spp., Cercospora apii, Cercospora beticola, Cercospora brassicicola, Cercospora kikuchii, Corynespora cassiicola, Cercospora zeae-maydis, Cercospora zeina, Dothistroma septosporum, Lecanosticta acicula, Mycocentrospora acerina, Passalora spp., Pseudocercospora fijiensis, Aureobasidium spp., Ophiosphaerella herpotricha, Parastagonospora nodorum, Diplodia tumefaciens, Alternaria alternate, Bipolaris maydis, Bipolaris oryzae, Bipolaris sacchari, Bipolaris victoriae, Curvulariaspp., Leptosphaerulina trifolii, Venturia inaequalis, Aspergillus spp., Aspergillus flavus, Blumeriagraminis, Frysiphe spp., Podosphaera leucotricha, Botrytis cinerea, Monilinia spp., Monilinia fructicola, Sclerotinia sclerotiorum, Amphilogia gyrosa, Cryphonectria parasitica, Diaporthe citri, Diaporthe helianthi, Diaporthe phaseolorum, Cytospora leucostoma, Colletotrichum spp., Colletotrichum coccodes, Colletotrichum gloeosporioides, Colletotrichum graminicola, Plectosphaerella cucumerina, Verticillium albo-atrum, Verticillium dahlia, Claviceps purpurea, Epichloe typhina, Trichoderma viride, Fusarium spp., Fusarium oxysporum, Fusarium solani, Fusarium graminearum, Nectria cinnabarina, Neonectria spp., Gaeumannomyces graminis, Pyricularia grisea, Pyricularia oryzae, Ceratocystis spp., Thielaviopsis basicola, Ophiostoma ulmi, Phyllachora graminis, Cronartium spp., Uromyces graminicola, Tranzschelia spp., Tilletia spp., Ustilago spp., Ustilago maydis, Peronospora spp., Hyalperonospora spp., Albugo spp., Phytophthora spp., Pythium spp., Aphanomyces spp., Magnaporthe oryzae, Puccinia, Blumeria graminis, Exserohilum turcicum, Mycosphaerella graminicola, Melampsora lini, Phakopsora pachyrhizi, Magnaporthiosis maydis (syn. Cephalosporium maydis or Harpophora maydis), Sarocladium strictum (syn. Cephalosporium acremonium or Acremontam strictum) and/or Rhizoctonia solani.

In some embodiments, a composition comprising the microorganism C. tofieldiae and/or extracts from said microorganism and/or filtrates from said microorganism applied to a plant and/or part thereof, and/or to a growth media, may increase resistance to a bacterial pathogen including, but not limited to, a bacterial pathogen from the family of Enterobacteriaceae, Pseudomonadaceae, Rhizobiaceae, Microbacteriaceae, Xanthomonadaceae, Rhizobiaceae, Corynebacteriaceae, Acetobacteraceae, Comamonadaceae, Bacillaceae, Burkholderiaceae, Micrococcaceae, Ralstoniaceae, Xanthomonadaceae, Spiroplasmataceae, Sphingomonadaceae, Acholeplasmataceae, Corynebacteriaceae, and/or Streptomycetaceae. In some embodiments, a composition of the disclosure applied to a plant and/or part thereof, and/or growth media, may increase resistance to a bacterial pathogen including, but not limited to, a bacterial pathogen from the genus of Erwinia spp., Dickeya spp., Pseudomonas spp., Xanthomonas spp., Agrobacterium spp., Rhizobium spp., Corynebacterium spp., Streptomyces spp., Pantoea spp., Serratia spp., Acetobacter spp., Acidovorax spp., Arthrobacter spp., Bacillus spp., Brenneria spp., Burkholderia spp., Clavibacter spp., Pectobacterium spp., Pantoea spp., Ralstonia spp., Xylella spp., Spiroplasma spp., Phytoplasma spp., and/or Sphingomonas spp.

In some embodiments, a composition comprising the microorganism C. tofieldiae and/or extracts from said microorganism and/or filtrates from said microorganism applied to a plant and/or part thereof, and/or to a growth medium, may increase resistance to a bacterial pathogen including, but not limited to, Erwinia amylovora, E. a carotovora var. chrysanthemi, D. dadanti, Pseudomonas tabaci, P. angulate, P. phaseolicola, P. lachrymans, P. pisi, P. fluorescens, P. glycinea. P. vesicatoria, P. savastanoi, P. syringae, P. solanacearum, Xanthamonas phaseoli, X. malvacearum, X. oryzae, X translucens, X. pruni, X. campestris, X. vasuclarum, Acidovorax avenae, Agrobacterium tumefaciens, A. rubi (=Rhizobium rubi), A. rhizogenes (=Rhizobium rhizogenes) and A. vitis (=Rhizobium vitis), Bacillus pumilus, Brenneria alni (=Erwinia alni), Clavibacter michiganensis, Pectobacterium carotovorum, Pantoea agglomerans, Ralstonia solanacearum, Corynebacterium insidiosum, C. sepedonicum, C. fascians, C. flacumfaciens, C. michiganense, Streptomyces scabies, S. ipomoeae, Pantoea agglomerans, Serratia marcescens, Streptomyces reticuliscabei, Acetobacter aceti, Spiroplasma citri Xylella fastidiosa, and/or Sphingomonas melonis.

As used herein, “disease resistance” or “disease tolerance” are used interchangeably and refer to a decrease in disease symptoms and/or a decrease in pathogen growth and reproduction in a plant and/or part thereof. In some embodiments, the percent (%) increase in resistance/tolerance to disease as compared to a control may be in a range from about 0.1% to about 100%. In some embodiments, the percent increase in resistance/tolerance to disease may be an increase in a range from about 0.1% to about 10%, 0.1% to about 30%, about 0.1% to about 50%, about 0.1% to about 80%, about 0.1% to about 90%, about 0.1% to about 95%, about 1% to about 10%, about 1% to about 20%, about 1% to about 40%, about 1% to about 50%, about 1% to about 75%, about 1% to about 95%, about 1% to about 100%, about 10% to about 20%, about 10% to about 40%, about 10% to about 50%, about 10% to about 70%, about 10% to about 80%, about 10% to about 90%, about 10% to about 100%, about 20% to about 40%, about 20% to about 75%, about 20% to about 90%, about 20% to about 95%, about 20% to about 100%, about 25% to about 50%, about 50% to about 75%, about 50% to about 95%, about 50% to about 100%, about 75% to about 90%, about 75% to about 100%, about 90% to about 95%, about 90% to about 100% or any value or range therein, as compared to a control. In some embodiments, the % increase in resistance/tolerance to disease may be about 0.1%, 0.5%, 1%, 1.5%, 2%, 2.5%, 3, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17, 5, 18%, 19%, 20%, 21%, 22%, 23%, 24%, 25%, 26%, 27%, 28%, 29%, 30%, 35%, 40%, 45%, or 50%, 55%, 60%, 65%, 70%, 75%, 80%, 85%, 90%, 95%, 96%, 97%, 98%, 99%or 100% or any value or range therein, as compared to a control.

In some embodiments, a method for increasing abiotic stress tolerance of a plant and/or part thereof is provided, the method comprising applying a composition comprising an effective amount of the microorganism C. tofieldiae and/or extracts from said microorganism and/or filtrates from said microorganism to a plant and/or part thereof, and/or to the growth media, thereby increasing the abiotic stress tolerance of the plant and/or part thereof as compared to a control plant and/or part thereof (e.g., a plant and/or part thereof to which the composition comprising the microorganism C. tofieldiae and/or extracts from said microorganism and/or filtrates from said microorganism has not been applied). In some embodiments, the method comprises applying the composition at least one time (e.g., about 1, 2, 3, 4, 5, 6, 7, 8, 9, 10, 11, 12 or more times, or any range or value therein). In some embodiments, the method comprises applying the composition at least two times (e.g., about 2, 3, 4, 5, 6, 7, 8, 9, 10, 11, 12 or more times, or any range or value therein).

In some embodiments, abiotic stress may include, but is not limited to, water stress, salinity (e.g., medium salinity (ECₑ=4-8 dSm⁻¹); high salinity (ECₑ>8 dSm⁻¹), flooding, freezing (e.g., about 0 °C or less), chilling or cold temperature (e.g., less than about 10-15 °C), heat or high temperatures (e.g., more than about 40 °C), high light intensity (e.g. more than about 10,000 foot candles), low light intensity (e.g. less than about 1000 foot candles), and/or ozone, and/or combinations thereof.

As used herein, “an ”increased tolerance to abiotic stress“ or ”increased resistance to abiotic stress” are used interchangeably and refer to the ability of a plant and/or part thereof, where the plant and/or part thereof, and/or growth media has been contacted with a composition comprising the microorganism C. tofieldiae and/or extracts from said microorganism and/or filtrates from said microorganism, to withstand a given abiotic stress better than a control plant and/or part thereof (i.e., a plant and/or part thereof, and/or growth media, that has been exposed to the same abiotic stress but has not been contacted with a composition comprising the microorganism C. tofieldiae and/or extracts from said microorganism and/or filtrates from said microorganism). Increased tolerance to abiotic stress can be measured using a variety of parameters including, but not limited to, the size and number of plants and/or parts thereof, and the like (e.g., number and size of fruits), the level or amount of cell division, the amount of floral abortion, the amount of sunburn damage, crop yield, and the like. Thus, in some embodiments of this disclosure, a plant and/or part thereof, where the plant and/or part thereof, and/or growth media have been contacted with a composition comprising the microorganism C. tofieldiae and/or extracts from said microorganism and/or filtrates from said microorganism, having increased tolerance to the abiotic stress would have, for example, increased fruit/seed number and/or weight as compared to a plant and/or part thereof exposed to the same stress but not having been contacted with said composition.

In some embodiments, the percent increase in resistance/tolerance to abiotic stress as compared to a control may be an increase in a range from about 0.1% to about 100%. In some embodiments, the percent increase in resistance/tolerance to abiotic stress may be in a range from about 0.1% to about 10%, 0.1% to about 30%, about 0.1% to about 50%, about 0.1% to about 80%, about 0.1% to about 90%, about 0.1% to about 95%, about 1% to about 10%, about 1% to about 20%, about 1% to about 40%, about 1% to about 50%, about 1% to about 75%, about 1% to about 95%, about 1% to about 100%, about 10% to about 20%, about 10% to about 40%, about 10% to about 50%, about 10% to about 70%, about 10% to about 80%, about 10% to about 90%, about 10% to about 100%, about 20% to about 40%, about 20% to about 75%, about 20% to about 90%, about 20% to about 95%, about 20% to about 100%, about 25% to about 50%, about 50% to about 75%, about 50% to about 95%, about 50% to about 100%, about 75% to about 90%, about 75% to about 100%, about 90% to about 95%, about 90% to about 100% or any value or range therein, as compared to a control. In some embodiments, the percent increase in resistance/tolerance to abiotic stress may be about 0.01%, 0.05%, 0.1%, 0.5%, 1%, 1.5%, 2%, 2.5%, 3, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17, 5, 18%, 19%, 20%, 21%, 22%, 23%, 24%, 25%, 26%, 27%, 28%, 29%, 30%, 35%, 40%, 45%, or 50%, 55%, 60%, 65%, 70%, 75%, 80%, 85%, 90%, 95%, 96%, 97%, 98%, 99% or 100% or any value or range therein, as compared to a control.

Another embodiment refers to the method of the disclosure wherein said microorganism is a strain of C. tofieldiae deposited under the deposit number CECT 20833 (C. tofieldiae Ct0861), CECT 20834, CECT 20835 or CECT 20836.

Three strains of C. tofieldiae were deposited on May 30, 2013 under the provisions of the Budapest Treaty in the International Depositary Authority “Colección Española de Cultivos Tipo” (CECT, for its Spanish acronym) at the address Pare Cientific Universitat de Valencia, Catedrático Agustín Escardino, 9. 46980, Paterna, (Valencia), Spain, and have been assigned deposit numbers CECT 20833 (C. tofieldiae Ct0861), CECT 20834 and CECT 20835. One strain of C. tofieldiae was deposited on May 7, 2013, under the provisions of the Budapest Treaty in the International Depositary Authority “Colecciín Española de Cultivos Tipo” (CECT) at the address Parc Cientific Universitat de Valencia, Catedrático Agustín Escardino, 9. 46980, Paterna, (Valencia), Spain, and has been assigned deposit number CECT 20836.

The deposits will irrevocably and without restriction or condition be available to the public upon issuance of a patent. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. 112. However, it should be understood that the availability of a deposit does not constitute a license to practice the subject disclosure in derogation of patent rights granted by government action. The deposit will be maintained without restriction in the CECT depository, which is a public depository, for a period of 30 years, or 5 years after the most recent request, or for the enforceable life of the patent, whichever is longer, and will be replaced if it ever becomes nonviable during that period.

The microorganism of the disclosure can be grown over a wide variety of natural or synthetic substrates. For example, it can be grown in different solid or liquid culture media, such as Potato/Dextrose/Agar (PDA) medium or broth (PDB), and it can be propagated by means of techniques being known per se by those skilled in the art. It can also grow over several natural sources, such as leaves from several plants, pollen grains, oat flower, potato, carrot, and cellulose. It can also grow over artificial sources such as paper or cardboard and polymers.

The culture medium can be under constant or occasional stirring during the culture, for example at approximately 1 rps or under culture conditions without any stirring or agitation. Furthermore, the culture temperature can be within a range from 20 to 30 °C.

Another embodiment refers to the method of the disclosure, wherein said microorganism is in the form of spores, hyphae, mycelium, or sclerotia.

Another embodiment refers to the method of the disclosure wherein said composition is applied to the substrate for the cultivation of said plant.

The substrate is preferably treated such that the microorganism of the disclosure is grown therein before said substrate is used for the plant's cultivation. Examples of substrate treatment include infusing a liquid into the substrate (by means of irrigation, injection or dripping), spraying, dusting or direct mixing with the substrate. The method of the disclosure also comprises treating a hydroponic medium, for hydroponic cultivation. The method of the disclosure consists of treating the substrate or hydroponic medium with a composition comprising the suitable concentration of mycelium and/or spores and/or any other part of the microorganism of the disclosure, the culture medium or filtrate according to the disclosure in liquid form and/or in solid form as granules or dust.

The method of the disclosure can be carried out with a composition including the microorganism of the disclosure either alone or formulated with inert ingredients. Examples of inert ingredients include fine powder or granules such as minerals, clays, bentonite, calcite, diatoms and organic materials such as corn powder or nut shell powder, synthetic organic materials such as urea, salts such as calcium carbonate and ammonium sulphate, synthetic inorganic materials such as silicon oxide; or liquid diluents such as aromatic hydrocarbons as xylene, alkyl benzene, methyl naphthalene, alcohols such as 2,5-propanol, ethylene glycol, glycol propylene, and ethylene glycol monoethyl ether, ketones such as acetone, cyclohexanone and isophorone, vegetable oils such as soya oil or cotton-seed oil, aliphatic hydrocarbons derived from petroleum, esters, dimethyl sulfoxide, acetonitrile and water. Examples of surfactants include anionic surfactants such as alkyl sulphate ester salts, alkyl aryl sulfonate salts, dialkylsulfosuccinate salts, polyoxyethylene alkyl aryl ether phosphate ester salts, lignosulphonate salts and formaldehyde polycondensates, and non-ionic surfactants such as polyoxyethylene alkyl aryl ethers, alkyl polyoxypropylene block copolymers, polyoxyethylene and fatty acid esters, and cationic surfactants such as alkyltrimethylammonium salts. Examples of other auxiliary formulation agents include water soluble polymers such as polyvinyl alcohol or polyvinylpyrrolidone, polysaccharides such as agar, acacia gum, alginic acid and salts thereof, carboxymethyl cellulose, xanthan gum, inorganic materials such as aluminum and magnesium silicate, preservatives, coloring agents, stabilizing agents such as isopropyl acid phosphate and BHT.

Therefore, another embodiment refers to the method of the disclosure wherein said composition comprises minerals, organic materials, organic compounds, inorganic compounds, liquid diluents, alcohols, ketones, vegetable oils, aliphatic hydrocarbons, esters, dimethyl sulfoxide, acetonitrile, water, anionic surfactants, non-ionic surfactants, cationic surfactants, water soluble polymers, polysaccharides, preservatives, coloring agents, and/or stabilizing agents. Particularly, said minerals are selected from the group consisting of clays, bentonite, calcite and diatoms, said organic materials are selected from the group consisting of corn powder or nut shell powder, said organic compound is urea, said inorganic compounds are selected from the group consisting of calcium carbonate, ammonium sulphate, silicon oxide, aluminum and magnesium silicate, said liquid diluents are selected from the group consisting of aromatic hydrocarbons, aliphatic hydrocarbons, alcohols, ketones, vegetable oils, esters, dimethyl sulfoxide, acetonitrile and water, said anionic surfactants are selected from the group consisting of alkyl sulphate ester salts, alkyl aryl sulfonate salts, dialkylsulfosuccinate salts, polyoxyethylene alkyl aryl ether phosphate ester salts, lignosulphonate salts and formaldehyde polycondensates, said non-ionic surfactants are selected from the group consisting of polyoxyethylene alkyl aryl ethers, alkyl polyoxypropylene block copolymers, polyoxyethylene and fatty acid esters, said cationic surfactants are alkyltrimethylammonium salts, said water soluble polymers are polyvinyl alcohol or polyvinylpyrrolidone, said polysaccharides are selected from the group consisting of agar, acacia gum, alginic acid, alginic acid salts, carboxymethyl cellulose and xanthan gum, and said stabilizing agents are isopropyl acid phosphate and BHT. Particularly, said aromatic hydrocarbons are selected from the group consisting of xylene, alkyl benzene, methyl naphthalene, said alcohols are selected from the group consisting of 2,5-propanol, ethylene glycol, glycol propylene, and ethylene glycol monoethyl ether, said ketones are selected from the group consisting of acetone, cyclohexanone and isophorone, and said vegetable oils are soya oil or cotton-seed oil.

Another embodiment refers to the method of the disclosure wherein said composition comprises organic irrigation salts, fertilizers, insecticides, nematicides, fungicides, bactericides and/or herbicides.

Another embodiment refers to the method of the disclosure wherein said composition is a liquid, a solid, a paste or a gel.

Another embodiment refers to the method of the disclosure wherein said composition is in the form of powder, pastille, tablet, granule or emulsifiable concentrate.

Another embodiment refers to the method of the disclosure wherein said composition is applied by spraying, atomization, immersion, irrigation or dusting.

Another embodiment refers to the method of the disclosure wherein said plant is selected from the group consisting of gymnospermae, monocotyledons and dicotyledoneae.

Another embodiment refers to the method of the disclosure wherein said plant is a monocotyledon plant.

Another embodiment refers to the method of the disclosure wherein said plant is a corn plant (Zea mays).

Another embodiment refers to the method of the disclosure wherein said plant is a tomato plant (Solanum lycopersicum).

In the present disclosure, the plant can be a natural or transgenic plant.

Another embodiment of the disclosure refers to a substrate for the cultivation of plants comprising the microorganism C. tofieldiae and/or extracts from said microorganism and/or filtrates from said microorganism.

The substrate for the cultivation of plants may comprise the spore, mycelium, or any other part of the microorganism of the disclosure or the culture medium or filtrate thereof, or any of the possible combinations of some of these components. The substrate may be liquid or solid. Examples of substrates for the cultivation of plants are natural or synthetic media being solidified, and those intended for plant cultivation especially in vitro. Other examples are soil, sand, humus or peat or a mixture thereof.

Another embodiment of the disclosure refers to a strain of the microorganism C. tofieldiae, deposited under the deposit number CECT 20833 (C. tofieldiae Ct0861), CECT 20834, CECT 20835 and CECT 20836.

The above summary is not intended to describe each illustrated example or every implementation of the subject matter hereof. The figures and the detailed description that follow more particularly exemplify various examples.

### BRIEF DESCRIPTION OF THE DRAWINGS

Subject matter hereof may be more completely understood in consideration of the following detailed description of various examples in connection with the accompanying figures, in which:

FIG. 1 is a graph indicating the relative abundance of each genus in maize grain at harvest with treatment of Colletotrichum tofieldiae strain Ct0861 compared to the control;

FIG. 2 is a graph indicating relative abundance of various amplicon sequence variant (ASV) sequences in the Aspergillus genus in grain samples from Colletotrichum tofieldiae strain Ct0861 inoculated plants as compared to controls;

FIG. 3 is a graph indicating quantification of Aspergillus flavus biomass using qPCR of the ITS DNA in maize grain samples that were colonized under LW (limited watering) and OW (optimal watering) conditions;

FIG. 4A is a representative picture of a dual culture bioassay between Colletotrichum tofieldiae strain Ct0861 and Aspergillus flavus CECT 2687 after 7 days facing Colletotrichum tofieldiae strain Ct0861 (left) and Aspergillus flavus CECT 2687 (right) in PDA plates;

FIG. 4B is a graph indicating mycelium growth of test plates and control plates during 7 days.

FIG. 5 is a graph indicating real Time PCR quantification of Botrytis cinerea actin gene DNA versus tomato ubiquitin DNA in leaf discs of mock and Colletotrichum tofieldiae strain Ct0861 inoculated plants, following protocol described in Haller et al. ; and

FIG. 6 is a graph indicating quantification of Fumonisin (B1+B2) in grain samples from untreated (Control) or Colletotrichum tofieldiae strain Ct0861 (Ct0861) treated maize plants.

While various examples are amenable to various modifications and alternative forms, specifics thereof have been shown by way of example in the drawings and will be described in detail. It should be understood, however, that the intention is not to limit the claimed inventions to the particular examples described. On the contrary, the intention is to cover all modifications, equivalents, and alternatives falling within the spirit and scope of the subject matter as defined by the claims.

### DETAILED DESCRIPTION

### Example 1: Measuring Relative Abundance of Fungal Genera in Grain

Colletotrichum tofieldiae strain Ct0861 was used to inoculate maize plants grown in open fields under optimal watering (OW) and limited watering (LW) conditions. The inoculated plants were compared to control plants for the relative abundance of ten fungal genera including Moesziomyces, Anthracocystis, Kurzmaniella, Meyerozyma, Cladosporium 1, Cladosporium 2, Fusarium, Penicillium, Aspergillus 1, and Aspergillus 2. Plants were treated with C. tofieldiaestrain Ct0861 or a control substance on the seeds and foliage and measured at harvest.

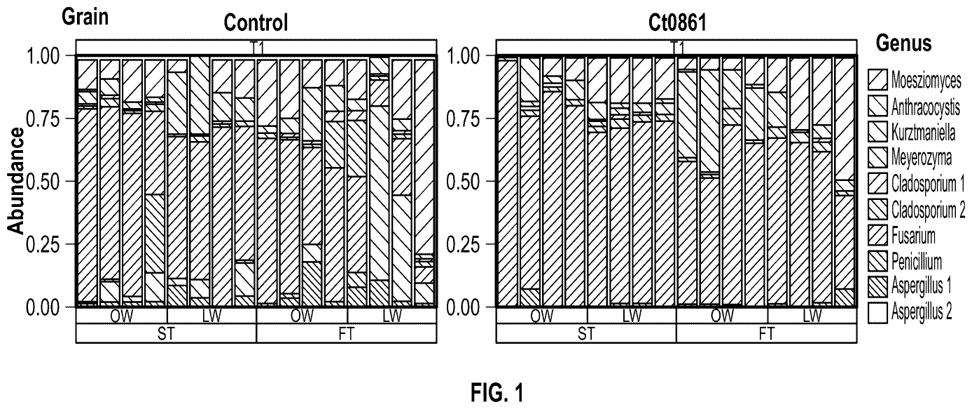

FIG. 1 shows the relative abundance of each genus in maize grain at harvest with treatment of Colletotrichum tofieldiae strain Ct0861 compared to the control. There is a significant reduction in the relative abundance of the Aspergillus genus from the inoculated plants compared with the control.

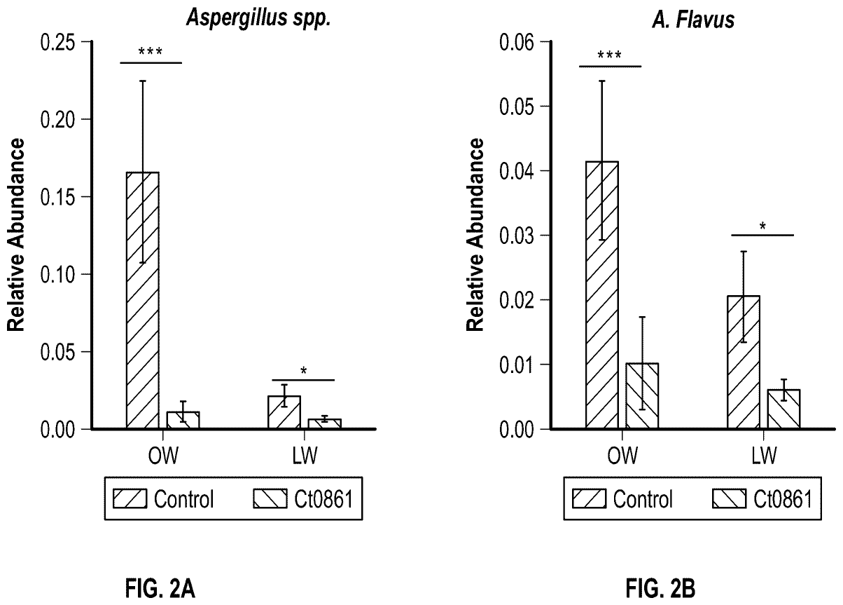

FIG. 2 shows relative abundance of various amplicon sequence variant (ASV) sequences in the Aspergillus genus in grain samples from Colletotrichum tofieldiae strain Ct0861 inoculated plants as compared to controls. The difference between the control and inoculated samples was especially pronounced in the OW regime, where Aspergillus spp. mean relative abundance reached 16.5% in grains from control plants and just 1% in grains from inoculated plants (t test, t−5.7, P<0.001). The mean relative abundance of Aspergillus spp. in inoculated plants under the LW regime was also significantly reduced when compared to controls (t test, t=2.5, P=0.02).

Within the Aspergillus spp. dataset, sequences associated with Aspergillus flavus and Aspergillus niger species were the most abundant, representing respectfully 71% and 26% of Aspergillus abundance in grain samples. A. flavus is an important mycotoxin producer, especially because of the production of aflatoxin (Pfliegler et al., 2020 Frontiers in Microbiology, 10, 2921). A. niger is a common contaminant in maize that produces the mycotoxins fumonisins B1, B2, and B4, and some strains may also synthesize ochratoxin A (Ismail, 2017 Food Control 73, 492-496). The relative abundance of A. flavus was significantly lower in grains from inoculated plants compared to those of controls (t test, t=4.2, P<0.001), with a reduction of 3% in relative abundance in OW conditions (WMW test, Z=2.7305, P=0.006) and a 1.4% reduction in LW conditions (t test, t=2.5, P=0.026). A. niger was present in the control grains in OW conditions at 12% relative abundance, which was reduced to 0.03% observed in the inoculated samples (WMW test, Z=3.4, P<0.001), while no differences were observed in LW conditions.

The relative abundance of Penicillium spp. in grain samples also showed significant differences between inoculated plants and controls with a hundredfold reduction in relative abundance (WMW test, Z=3.1, P=0.002).

### Example 2: Real Time PCR Biomass Quantification of A. flavus

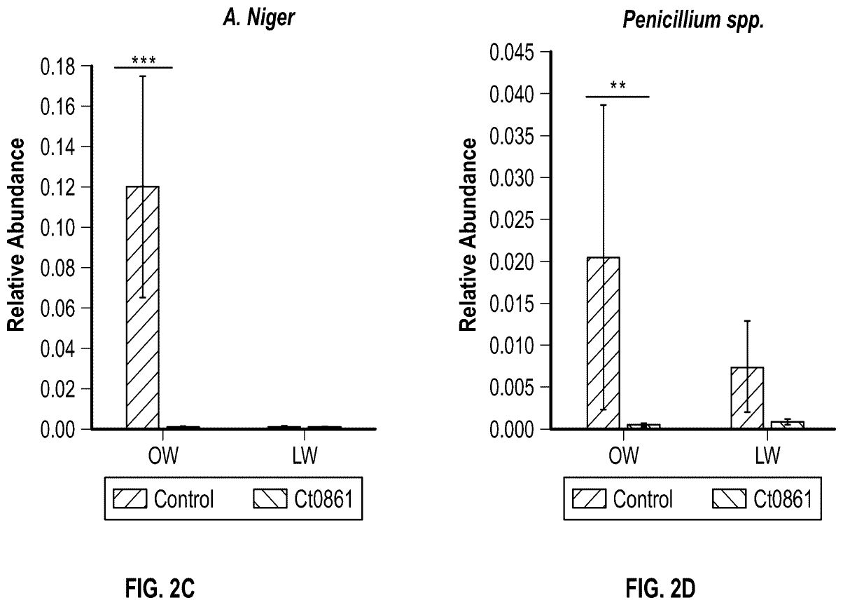

In Mideros et al. 2009 (Plant Disease-93. 1163-1170), it is shown that Aspergillus flavus biomass in maize grains is strongly correlated with aflatoxin concentration. Using the protocol described in Mideros et al. 2009, which is herein incorporated by reference in its entirety, Real Time PCR quantification of A. flavus from maize grains was performed. Results of the Real Time PCR confirmed results from Example 1 for A. flavus in OW conditions, showing significant differences between treatments (FIG. 3). Mideros et al. 2009 showed, that the amount of A. flavus measured using the protocol described herein is proportional to aflatoxins concentration. Thus, in this example, the aflatoxin concentration would be proportionally reduced in samples inoculated with Colletotrichum tofieldiae strain Ct0861.

FIG. 3 shows quantification of A. flavus biomass using qPCR of the ITS DNA in maize grain samples that were colonized under LW and OW conditions. In OW conditions, inoculation with Colletotrichum tofieldiae strain Ct0861 significantly decreased A. flavus compared to the control.

### Example 3: Evaluation of Possible Direct Effect of Colletotrichum tofieldiae Strain Ct0861 on Aspergillus flavus

In order to evaluate if the effect of C. tofieldiae strain Ct0861 is due to direct competence, antibiosis, or predation on A. flavus, a dual culture bioassay was conducted using A. flavus aflatoxin producing stain CECT 2687. The protocol described in Moreno-Gavira et al. 2021 (J. Fungi 7, 415) was used for the assay, which is herein incorporated by reference in its entirety.

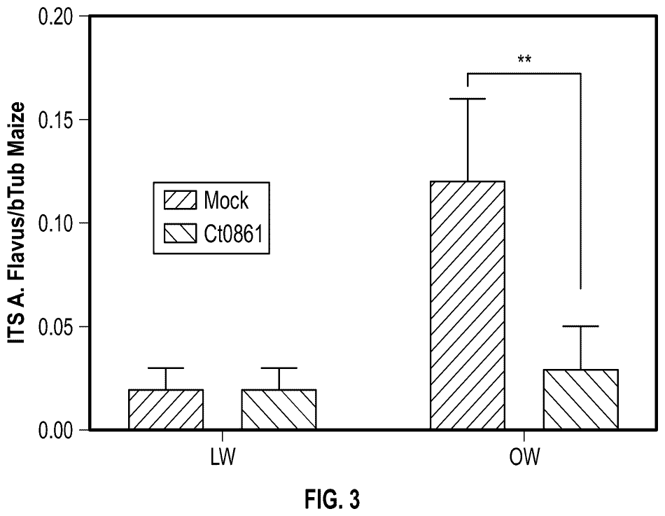

After 7 days of co-cultivation, there was no reduction in the growth of A. flavus CECT 2687, as shown in FIG. 4. Control plates where two plugs of A. flavus were faced showed a higher growth inhibition than test plates where A. flavus CECT 2687 was faced against C. tofieldiae strain Ct0861. Additionally, C. tofieldiae strain Ct0861 DNA was not detected in the maize samples used for A. flavus biomass quantification, indicating that the effect observed is not likely due to a direct fungal interaction.

### Example 4. Less Growth of the Fungus Botrytis cinerea in Leaves of Tomato Plants Root Inoculated With Colletotrichum tofieldiae.

Tomato (Solanum lycopersicum) seeds were sterilized and germinated in filter paper. One week old seedlings were inoculated with Colletotrichum tofieldiae by dipping the roots in a solution with 10⁶ conidia/ml of strain Ct0861 or in water for mock treatment. One month old tomato plants were inoculated with Botrytis cinerea by applying 2 drops of 10μ of 10⁶ conidia/ml. After 72 hours, 5 mm diameter leaf discs were punched from each Botrytis lesion, and fungal vs plant DNA was quantified by Real Time PCR following the protocol described in Haller et al. (2020) Front. Plant Sci. 11:594827

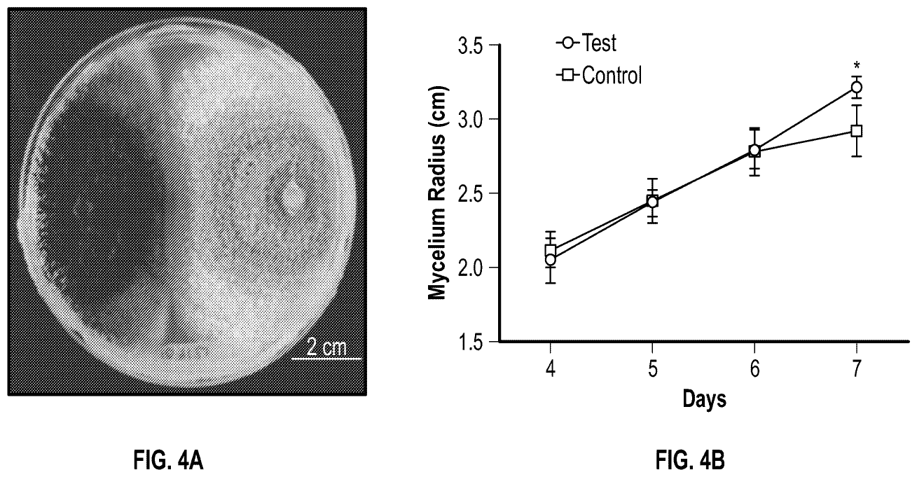

FIG. 5 shows that Botrytis accumulation in lesions from Ct0861 inoculated vs Mock plants is reduced by 49%.

### Example 5: Measuring the Mycotoxin Fumonisin in Grain

Colletotrichum tofieldiae strain Ct0861 was applied to maize plants at planting in small plot replicated field trials and compared to an untreated control. The grain from C. tofieldiae strain Ct0861 and the untreated control plants was harvested and analyzed for the concentration of the mycotoxin Fumonisin (B1+B2). Fumonisin B1 and B2 were determined after extraction with acidified acetonitrile and detection by liquid chromatography combined with tandem mass spectrometry (LC-MS/MS) in which two mass analyzers are operated in series, as known to one of ordinary skill in the art.

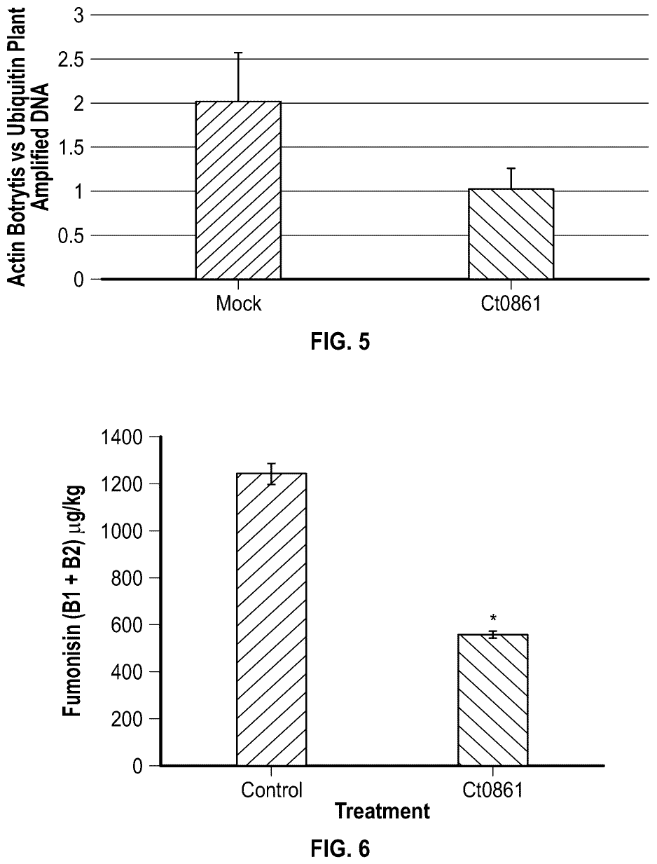

FIG. 6 shows the concentration of Fumonisin (B1+B2) in maize grain at harvest with treatment of C. tofieldiae strain Ct0861 compared to the control. There is a significant reduction in Fumonisin (B1+B2) in the inoculated plants compared with the control.

The results of Examples 1, 2 and 4 indicate an effect of Colletotrichum tofieldiae strain Ct0861 in reducing the growth of toxigenic fungi, especially from the Aspergillus genera in maize grains, and Botrytis cinerea in tomato leaves. Example 5 shows that this reduction can be correlated with a reduction in aflatoxin content in maize grains. This effect is not likely due to a direct interaction between C. tofieldiae strain Ct0861 and A. flavus in the grain (Example 3) but rather due to the induction of plant resistance, a possible feature of C. tofieldiae strain Ct0861. It is known that C. tofieldiae strain Ct0861 in a plant's roots triggers Arabidopsis immune pathways in the leaves that rely on glucosinolate metabolism as well as salicylic acid and ethylene signaling (Frerigmann et al. 2021 Molecular Plant-Microbe Interactions® 34:5, 560-570). In maize, it has been shown that salicylic acid and steroid signaling pathways are involved in a long-lasting primed state in maize triggered by Tichoderma harziamm, which activates defense responses in maize silks that may protect grains from fungal infection (Agostini et al. 2019 Molecular Plant-Microbe Interactions® 32:1, 95-106). In tomato, induced resistance by irrigating with hexaonic acid reduced the growth of B. cinerea inoculated on leaves (Vicedo et al. 2009. MPMI Vol. 22, No. 11, 2009, pp. 1455-1465. Doi: 10.1094/MPMI). There may be a similar effect produced by C. tofieldiae strain Ct0861. Zahra et al. (2023) Physiological and Molecular Plant Pathology 127:102125, incorporated herein by reference in its entirety, indicate that the signaling and transcriptional regulation in the induced resistance process is involved, as well, in the tolerance to abiotic stress (see page 7).

Various embodiments of systems, devices, and methods have been described herein. These embodiments are given only by way of example and are not intended to limit the scope of the claimed disclosures. It should be appreciated, moreover, that the various features of the embodiments that have been described may be combined in various ways to produce numerous additional embodiments. Moreover, while various materials, dimensions, shapes, configurations, and locations, etc. have been described for use with disclosed embodiments, others besides those disclosed may be utilized without exceeding the scope of the claimed disclosures.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

As used in the description of the disclosure and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The terms “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 5%, 10%, 15%, 20%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount.

As used herein, “a plant” can be, but is not limited to, any monocotyledonous and dicotyledonous plant, and any annual and perennial dicotyledonous and monocotyledonous plant. Example plants include, but are not limited to, those of the genera Glycine, Vitis, Asparagus, Populus, Pennisetum, Lolium, Oryza, Zea, Avena, Hordeum, Secale, Triticum, Helianthus, Gossypium, Medicago, Pisum, Acer, Actinidia, Abelmoschus, Agropyron, Allium, Amaranthus, Apium, Arachis, Asparagus, Beta, Brassica, Camellia, Canna, Capsicum, Carex, Carica papaya, Carya, Castanea, Cinnamomum, Citrullus, Citrus, Cocos, Coffea, Colocasia, Cola, Coriandrum, Corylus, Crataegus, Crocus, Cucurbita, Cucumis, Cynara, Daucus, Desmodium, Dimocarpus, Dioscorea, Diospyros, Echinochloa, Elaeis, Eleusine, Eriobotrya, Eugenia, Fagopyrum, Fagus, Ficus, Fortunella, Fragaria, Ginkgo, Hemerocallis, Hibiscus, Ipomoea, Juglans, Lactuca, Lathyrus, Lens, Linum, Litchi, Lotus, Lupinus, Luzula, Malus, Malpighia, Mammea, Mangifera,Manihot, Manilkara, Medicago, Melilotus, Mentha, Miscanthus, Musa, Nicotiana, Olea, Opuntia, Ornithopus, Panicum, Passiflora, Persea, Phaseolus, Pinus, Pistacia, Pisum, Poa, Prosopis, Prunus, Quercus, Raphanus, Rheum, Ribe, Rubus, Sambucus, Secale, Sesamum, Sinapis, Solanum, Sorghum, Spinacia, Tamarindus, Theobroma, Trifolium, Tropaeolum, Vaccinium, Vigna, Vitis, Zizania or Ziziphus Sorghum, Saccharum and Lycopersicum, or the class Liliatae. In some embodiments, a plant or part thereof is from the genera Glycine, Vitis, Asparagus, Populus, Pennisetum, Lolium, Oryza, Zea, Avena, Hordeum, Secale, Triticum, Sorghum, Saccharum and Lycopersicum, or the class Liliaceae.

As used herein, a “part thereof” includes, but is not limited to, plant reproductive tissues (e.g., petals, sepals, stamens, pistils, receptacles, anthers, pollen, flowers, fruits, flower buds, ovules, seeds, embryos, nuts, kernels, bulbs, ears, cobs and husks); plant vegetative tissues (e.g., petioles, stems, roots, root hairs, root tips, pith, coleoptiles, stalks, shoots, branches, bark, apical meristem, axillary bud, cotyledon, hypocotyls, and leaves); plant vascular tissues (e.g., phloem and xylem); specialized plant cells such as epidermal cells, parenchyma cells, collenchyma cells, sclerenchyma cells, stomata, guard cells, cuticle, mesophyll cells; callus tissue; and cuttings. It also includes plant cells, including plant cells that are intact in plants and/or parts of plants, plant protoplasts, plant tissues, plant organs, plant cell tissue cultures, plant calli, plant clumps, and the like.

A “growth media” or “growing media”, as used herein includes, but is not limited to soil, synthetic growth media and/or aqueous solution in which the plant is planted (e.g., a hydroponic solution). A growing media may be treated/contacted with a composition comprising C. tofieldiae before and/or after sowing seedlings or seeds including spraying or irrigating the growth medium with a composition comprising C. tofieldiae. For example, a composition comprising C. tofieldiae formulated into a liquid or solid formulation, such as a granule, may be applied to soil around seedlings prior to, during and/or after sowing the seedlings. In some aspects, both a growth medium and the plant or plant part planted or sowed in the same may be treated/contacted with a composition comprising C. tofieldiae.

The term “abiotic stress” as used herein refers to outside, nonliving, factors which can cause harmful effects to plants. Thus, as used herein, abiotic stress includes, but is not limited to, cold temperature that results in freezing, chilling, heat or high temperatures, water stress, high light intensity, low light intensity, salinity, flooding (excess water/water-logging), ozone, and/or combinations thereof. Parameters for the abiotic stress factors are species specific and even variety specific and therefore vary widely according to the species/variety exposed to the abiotic stress. Thus, while one species may be severely impacted by a high temperature of 23 °C, another species may not be impacted until at least 30 °C, and the like. Temperatures above 30 °C result in dramatic reductions in the yield of most plants. This is due to reductions in photosynthesis that begin at approximately 20-25 °C, and the increased carbohydrate demands of crops growing at higher temperatures. The critical temperatures are not absolute but vary depending upon such factors as the acclimatization of plants to prevailing environmental conditions. In addition, because most plants are exposed to multiple abiotic stresses at one time, the interaction between the stresses affects the response of the plant. Thus, the particular parameters for high/low temperature, light intensity, water stress and the like, which impact plant productivity will vary with species, variety, degree of acclimatization and the exposure to a combination of environmental conditions.

For purposes of interpreting the claims, it is expressly intended that the provisions of 35 U.S.C. § 112(f) are not to be invoked unless the specific terms “means for” or “step for” are recited in a claim.

1. A method for reducing plant disease comprising:
contacting a plant with a composition comprising the microorganism Colletotrichum tofieldiae or extracts of the microorganism or filtrates of the microorganism.

2. A method for reducing abiotic stress comprising:
contacting a plant with a composition comprising the microorganism Colletotrichum tofieldiae or extracts of the microorganism or filtrates of the microorganism.

3. A method for reducing the colonization of fungi on a plant or a plant-derived product comprising:
contacting a plant with a composition comprising the microorganism Colletotrichum tofieldiae or extracts of the microorganism or filtrates of the microorganism.

4. A method for reducing mycotoxins on a plant or a plant-derived product comprising:
contacting a plant with a composition comprising the microorganism Colletotrichum tofieldiae or extracts of the microorganism or filtrates of the microorganism.

5. The method of claim 1, wherein the microorganism is a Colletotrichum tofieldiae strain deposited under deposit numbers CECT 20833, CECT 20834, CECT 20835 or CECT 20836.

6. The method of claim 1, wherein the microorganism is in the form of spores, hyphae, mycelium or sclerotia.

7. The method of claim 3, wherein the fungi comprise any of Alternaria sp., Phytophthora spp., Penicillium spp., Colletotrichum spp., Fusarium spp., Botrytis spp., Cephalosporium spp., and Aspergillus spp.

8. The method according to claim 1, wherein the composition is applied to the seeds of the plant.

9. The method according to claim 1, wherein the composition is applied to the aerial parts of the plant.

10. The method according to claim 1, wherein the composition is applied to the roots of the plant or to other underground parts of the plant.

11. The method according to claim 1, wherein the composition is applied to the soil, field, or other matrix of the plant or to other underground parts of the plant.

12. The method according to claim 1, wherein the composition is applied to a substrate for the cultivation of the plant.

13. The method according to claim 1, wherein the composition is applied to the grain of the plant.

14. The method according to claim 1, wherein the composition comprises minerals, organic materials, organic compounds, inorganic compounds, liquid diluents, alcohols, ketones, vegetable oils, aliphatic hydrocarbons, esters, dimethyl sulfoxide, acetonitrile, water, anionic surfactants, nonionic surfactants, cationic surfactants, water-soluble polymers, polysaccharides, preservatives, coloring agents, and/or stabilizers.

15. The method according to claim 14, wherein in that the minerals are selected from the group consisting of clays, bentonite, calcite and diatomaceous, the organic materials are selected from the group consisting of maize powder or nut skin powder, the organic compound is urea, the inorganic compounds are selected from the group consisting of calcium carbonate, ammonium sulfate, silicon oxide, aluminum and magnesium silicate, the liquid diluents are selected from the group consisting of aromatic hydrocarbons, aliphatic hydrocarbons, alcohols, ketones, vegetable oils, esters, dimethyl sulfoxide, acetonitrile and water, the anionic surfactants are selected from the group consisting of salts of alkyl sulfate esters, alkyl aryl sulfonate salts, dialkyl sulfosuccinate salts, polyoxyethylene alkyl aryl phosphate ether ester salts, lignosulfonate salts, and formaldehyde polycondensates, the nonionic surfactants are selected from the group consisting of polyoxyethylene alkyl aryl ethers, alkyl polyoxypropylene block copolymers, polyoxyethylene, and fatty acid esters, the cationic surfactants are alkyl trimethyl ammonium salts, the water-soluble polymers are polyvinyl alcohol or polyvinylpyrrolidone, the polysaccharides are selected from the group consisting of agar, acacia, alginic acid, alginic acid salts, carboxy methyl cellulose, and xanthan gum and the stabilizers are isopropyl acid phosphate or BHT.

16. The method according to claim 15, wherein the aromatic hydrocarbons are selected from the group consisting of xylene, alkyl benzene, methyl naphthalene, the alcohols are selected from the group consisting of 2-5 propanol, ethylene glycol, propylene glycol and ethylene glycol mono ethyl ether, the ketones are selected from the group consisting of acetone, cyclohexanone and isophorone and the vegetable oils are soybean oil or cottonseed oil.

17. The method according to claim 1, wherein the composition comprises fertigation salts, fertilizers, insecticides, nematicides, fungicides, bactericides, or herbicides.

18. The method according to claim 1, wherein the composition comprises a beneficial microbe, optionally wherein the beneficial microbe is Bacillus spp, Pseudomonas spp, Azotobacter spp, Rhizobium spp, Azorhizobium spp, Chaetomium spp, Streptomyces spp. Trichoderma spp., and/or mycorrhizal fungi.

19. The method according to claim 1, wherein the composition is a liquid, a solid, a paste, or a gel.

20. The method according to claim 1, wherein the composition is a powder, pill, tablet, granulate, or emulsifiable concentrate.

21. The method according to claim 1, wherein the composition is applied by spray, pulverization, immersion, irrigation, basal application, top dressing, side dressing, broadcasting, localized combined placement, banded or spot placement, liquid or dry application in starter solution, liquid or dry application in irrigation, liquid or dry application in injection, liquid or dry application in soil, liquid or dry application to foliage, or dusting.

22. The method according to claim 1, wherein plant or the plant-derived product is a gymnosperm or dicotyledonous plant.

23. The method according to claim 1, wherein the plant or a plant-derived product is from the Brassicaceae family.

24. The method according to claim 1, wherein the plant or a plant-derived product is from a monocotyledonous plant.

25. The method according to claim 1, wherein the plant or a plant-derived product is from maize (Zea mays).

26. The method according to claim 1, wherein the plant or a plant-derived product is from tomato (Solanum lycopersicum).

27. A method for reducing mycotoxin contamination on a plant or a plant-derived product, comprising:
contacting the plant with a composition comprising the microorganism Colletotrichum tofieldiae or extracts of the microorganism or filtrates of the microorganism.

28. The method of claim 27, wherein the microorganism is in the form of spores, hyphae, mycelium or sclerotia.

29. The method of claim 27, wherein the microorganism is a Colletotrichum tofieldiae strain deposited under deposit numbers CECT 20833, CECT 20834, CECT 20835 or CECT 20836.

30. The method of claim 27, wherein the mycotoxins include aflatoxins, fumonisins, ochratoxins, citrinin, patulin, sterigmatocystin, gliotoxin, zearalenone, trichothecenes, nivalenol, or deoxynivalenol.

31. A kit for reducing the disease, abiotic stress, colonization of fungi or mycotoxins on a plant or a plant-derived product, comprising:
a composition comprising the microorganism Colletotrichum tofieldiae; and
instructions for applying the composition to the plant or a plant-derived product.

32. The kit of claim 31, wherein the composition comprises extracts or filtrates of the microorganism Colletotrichum tofieldiae.

33. The kit of claim 31 wherein the microorganism is in the form of spores, hyphae, mycelium or sclerotia.

34. The kit of claim 31, wherein the microorganism is a Colletotrichum tofieldiae strain deposited under deposit numbers CECT 20833, CECT 20834, CECT 20835 or CECT 20836.

35. A method for reducing plant disease comprising:
providing a composition of the microorganism Colletotrichum tofieldiae or extracts of the microorganism or filtrates of the microorganism.

36. A method for reducing abiotic stress comprising:
providing a composition of the microorganism Colletotrichum tofieldiae or extracts of the microorganism or filtrates of the microorganism.

37. A method for reducing the colonization of fungi on a plant or a plant-derived product comprising:
providing a composition of the microorganism Colletotrichum tofieldiae or extracts of the microorganism or filtrates of the microorganism.

38. A method for reducing the mycotoxins on a plant or a plant-derived product comprising:
providing a composition of the microorganism Colletotrichum tofieldiae or extracts of the microorganism or filtrates of the microorganism.
